(19) United States
(12) Patent Application Publication
(10) Pub. No.: US 2009/0280068 A1
(43) Pub. Date: Nov. 12, 2009

(54) POLYMORPH OF 8-HYDROXY-5-[(1R)-1-HYDROXY-2-[[(1R)-2-(4-METHOXYPHENYL)-1-METHYLETHYL]AMINO]ETHYL]-2(1H)-QUINOLINONE MONOHYDROCHLORIDE

(75) Inventors: Fausto PIVETTI, Parma (IT); Emilio LUTERO, Parma (IT)

Correspondence Address: OBLON, SPIVAK, MCCLELLAND MAIER & NEUSTADT, L.L.P., 1940 DUKE STREET, ALEXANDRIA, VA, 22314, US

(73) Assignee: Chiesi Farmaceutici S.p.A., Parma (IT)

(21) Appl. No.: 12/436,368

(22) Filed: May 6, 2009

(30) Foreign Application Priority Data
May 7, 2008 (EP) 08155799.3

(51) Int. Cl. A61K 9/12; A61K 31/4704; C07D 215/227; A61P 11/06

(52) U.S. Cl. 424/45; 514/312; 546/157

(57) ABSTRACT

Crystal form D of 8-hydroxy-5-[(1R)-1-hydroxy-2-[[(1R)-2-(4-methoxyphenyl)-1-methylethyl]-amino]ethyl]-2(1H)-quinolinone monohydrochloride is highly crystalline, easy to prepare, and stable.

### CROSS REFERENCES TO RELATED APPLICATIONS

This application claims priority to European Patent Application No. 08155799.3, filed on May 7, 2008, which is incorporated herein by reference in its entirety.

### BACKGROUND OF THE INVENTION

1. Field of the Invention

The present invention relates to novel polymorphic crystal forms of 8-hydroxy-5-[(1R)-1-hydroxy-2-[[(1R)-2-(4-methoxyphenyl)-1-methylethyl]-amino]ethyl]-2(1H)-quinolinone monohydrochloride. The present invention also relates to processes for the preparation of such a crystal form, pharmaceutical compositions which contain such a crystal form, and methods for treating and/or preventing certain conditions by administering such a crystal form.

2. Discussion of the Background

8-Hydroxy-5-[(1R)-1-hydroxy-2-[[(1R)-2-(4-methoxyphenyl)-1-methylethyl]amino]ethyl]2(1H)-quinolinone monohydrochloride (I):

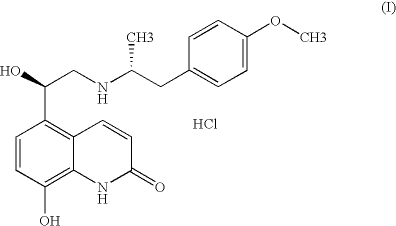

has been described in EP 147719 as a bronchodilator haring a potent beta-2-adrenoceptor stimulating action. The compound, which has also been referred to in the literature with the codes TA 2005 and CHF 4226, may be prepared in a prevalently amorphous form in accordance with the method given in Example 4 of EP 147719.

This compound, hereinafter referred to as “CHF 4226”, has been investigated for use as a medicament for treating inflammatory or obstructive airways diseases. However, for preparing suitable pharmaceutical compositions, it is important that the compound exist as a thermodynamically stable crystal form. It is also important that the compound have good handling qualities and can be easily obtained on a commercial scale.

A thermodynamically stable crystal form of CHF 4226, hereinafter referred to as form A, has been disclosed and closely characterized in WO 2005/089760, but said form can be obtained in an adequate pharmaceutical level of chemical purity and crystallinity only by applying specific crystallization conditions.

Therefore, it would be advantageous to provide further thermodynamically stable crystal forms of CHF 4226, which are easy to obtain by crystallization, and characterized by a high level of chemical purity and crystallinity as well as good handling qualities for pharmaceutical use.

### SUMMARY OF THE INVENTION

Accordingly, it is one object of the present invention to provide novel crystal forms of CHF 4226.

It is another object of the present invention to provide novel crystal forms of CHF 4226, which are thermodynamically stable.

It is another object of the present invention to provide novel crystal forms of CHF 4226, which are easy to obtain by crystallization.

It is another object of the present invention to provide novel crystal forms of CHF 4226, which are characterized by a high level of chemical purity.

It is another object of the present invention to provide novel crystal forms of CHF 4226, which are characterized by a high level of crystallinity.

It is another object of the present invention to provide novel crystal forms of CHF 4226, which exhibit good handling qualities for pharmaceutical use.

It is another object of the present invention to provided novel methods for preparing such a crystal form.

It is another object of the present invention to provide novel pharmaceutical compositions which contain such a crystal form.

It is another object of the present invention to provide novel methods of treating and/or preventing certain conditions and/or diseases by administering such a crystal form.

These and other objects, which will become apparent during the following detailed description, have been achieved by the inventors' discovery of a novel polymorphic crystal form of 8-hydroxy-5-[(1R)-1-hydroxy-2-[[(1R)-2-(4-methoxyphenyl)-1-methylethyl]amino]ethyl]-2(1H)-quinolinone monohydrochloride (CHF 4226), designated hereinafter as crystal form D.

Said polymorph is a thermodynamically stable anhydrous form, and is characterized by high level of chemical purity and crystallinity as well as good handling characteristics for the preparation of pharmaceutical compositions.

Crystal form D may be selectively produced by crystallization from appropriate solvents and conditions and is distinguishable based upon its characteristic peaks in the X-ray powder diffraction (XRPD) pattern, and its characteristic melting range.

Accordingly, the invention present invention also provides processes for the preparation of said form comprising crystallization or re-crystallization from appropriate solvents.

The present invention further provides pharmaceutical compositions comprising CHF 4226 crystal form D herein described, and to its use as a medicament.

The polymorph of the present invention is preferably administered by inhalation for the prevention and/or treatment of an inflammatory or obstructive respiratory disease such as asthma or chronic obstructive pulmonary disease (COPD).

Accordingly, in a further aspect, the present invention provides methods for the prevention and/or treatment of an inflammatory or obstructive respiratory disease such as asthma or chronic obstructive pulmonary disease (COPD) by administering CHF 4226 crystal form D.

In a still further aspect, the present invention provides a method of preventing and/or treating an inflammatory or obstructive respiratory disease such as asthma or chronic obstructive pulmonary disease (COPD), which comprises the inhalatory administration of an effective amount of CHF 4226 crystal form D described before.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a differential scanning calorimetry (DSC) thermal trace of crystal form D.

FIG. 2 is an X-ray powder diffraction (XRPD) pattern of crystal form D.

FIG. 3 is a comparative DSC thermal trace of crystal form A.

FIG. 4 is a comparative XRPD pattern of crystal form A.

### DETAILED DESCRIPTION OF THE PREFERRED EMBODIMENTS

Unless defined otherwise, all technical and scientific terms used herein have the same meaning as it is commonly understood by one of skill in the art to which this subject matter belongs.

As used herein, the term “amorphous” describes a non-ordered solid state characterized by a diffused X-ray powder diffraction with no sharp peaks.

As used herein, the term “prevalently amorphous”, describes a non-ordered solid state characterized by a diffused X-ray powder diffraction pattern with few sharp peaks.

As used herein, “polymorphism” is the ability of a compound to crystallize into more than one distinct crystal species. Polymorphs (or crystalline modifications) have an identical chemical structure but quite different physicochemical properties.

As used herein, “an effective amount” of a compound for treating a particular disease is an amount that is sufficient to ameliorate, or in some manner reduce the symptoms associated with the disease.

As used herein, the term “thermodynamically stable” refers to a polymorphic form that, during storage under long-term conditions (25° C., 60% relative humidity), substantially does not convert into another crystal form for a pharmaceutically acceptable period of time (at least 3 months, preferably 6 months, more preferably 1 year).

As used herein, the term “high level of chemical purity” refers to a polymorph wherein the total amount of readily detectable impurities as determined by standard methods of analysis, such as thin layer chromatography (TLC) or high performance liquid chromatography (HPLC), used by those of skill in the art to assess such purity, is less than 5%, advantageously less than 2.5%, preferably less than 1.0, more preferably less than 0.5% w/w.

As used herein, the term “high level of crystallinity” refers to a polymorph wherein the percentage of crystallinity is equal to or higher than 90%, preferably higher than 95% w/w as determined by standard methods of analysis used by those of skill in the art, such as X-ray powder diffraction or microcalorimetry.

Thus, in a first embodiment, the present invention provides a novel thermodynamically stable crystal form of CHF 4226 having a high level of chemical purity and crystallinity, designated crystal form D. Crystal form D is an anhydrous crystalline powder which can be characterized in a variety of ways.

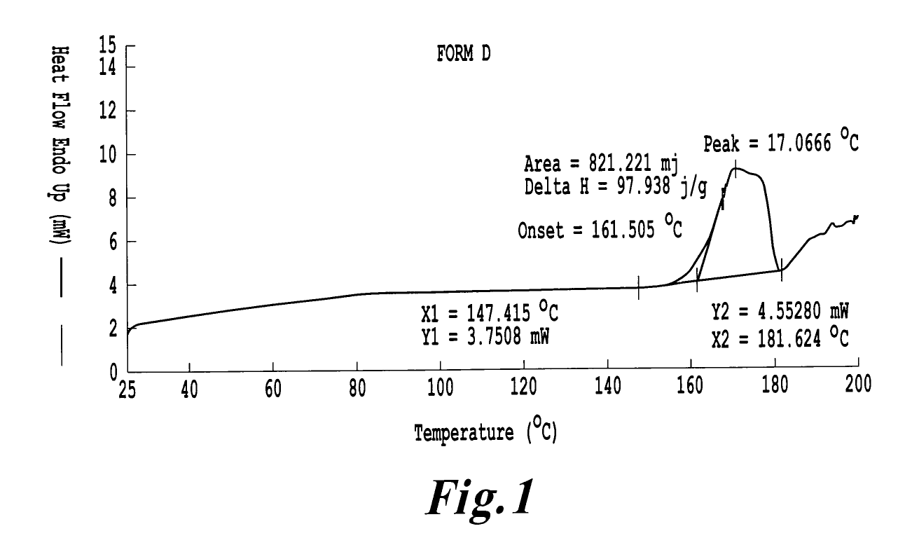

Its thermal trace, shown in FIG. 1, exhibits a melting peak at 170.7° C.

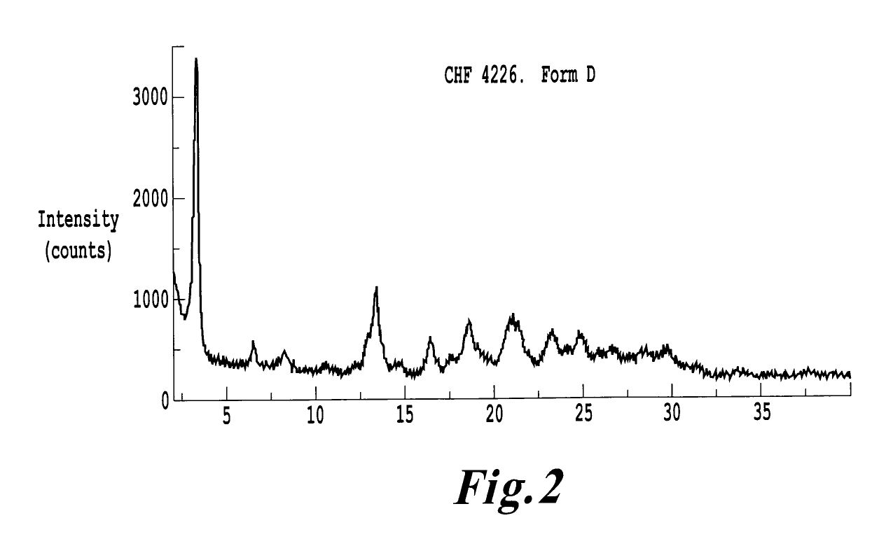

A PXRD pattern for Crystal form D using Cu—Kα radiation is shown in FIG. 2 and reported below in Table 1.

TABLE 1

| Pos. [°2θ] | Height [cts] | FWHM [°2θ] | d-spacing [Å] | Rel. Int. [%] |
|---|---|---|---|---|
| 3.3873 | 2707.68 | 0.1506 | 26.08407 | 100.00 |
| 6.5379 | 196.48 | 0.2007 | 13.51986 | 7.26 |
| 8.3164 | 139.44 | 0.2342 | 10.63210 | 5.15 |
| 10.4880 | 66.40 | 0.2676 | 8.43501 | 2.45 |
| 12.1570 | 67.77 | 0.2007 | 7.28048 | 2.50 |
| 12.8490 | 313.23 | 0.2007 | 6.88991 | 11.57 |
| 13.4176 | 767.41 | 0.1840 | 6.59916 | 28.34 |
| 14.7949 | 63.27 | 0.4684 | 5.98777 | 2.34 |
| 16.4369 | 279.66 | 0.1673 | 5.39314 | 10.33 |
| 17.5646 | 63.31 | 0.3346 | 5.04934 | 2.34 |
| 18.6855 | 366.75 | 0.2676 | 4.74890 | 13.54 |
| 21.0160 | 401.78 | 0.4684 | 4.22726 | 14.84 |
| 21.4718 | 350.19 | 0.2676 | 4.13855 | 12.93 |
| 23.3344 | 260.20 | 0.2676 | 3.81224 | 9.61 |
| 24.8162 | 209.90 | 0.4684 | 3.58787 | 7.75 |
| 26.7332 | 90.83 | 0.4015 | 3.33478 | 3.35 |
| 28.4251 | 74.92 | 0.4015 | 3.14003 | 2.77 |
| 29.8453 | 124.85 | 0.4684 | 2.99376 | 4.61 |
| 33.8028 | 36.53 | 0.6691 | 2.65177 | 1.35 |
| 37.7254 | 34.89 | 0.5353 | 2.38458 | 1.29 |

Thus, in a second embodiment, the present invention provides crystal form D which has the following characteristic diffraction peaks at 2θ in angular degrees using Cu—Kα radiation:

about 3.39,

about 13.42,

about 18.69,

about 21.02, and

about 21.47.

In a third embodiment, the present invention provides crystal form D which has the following characteristic diffraction peaks at 2θ in angular degrees using Cu—Kα radiation:

about 3.39,

about 12.85,

about 13.42,

about 18.69,

about 21.02, and

about 21.47.

In a fourth embodiment, the present invention provides crystal form D which has the following characteristic diffraction peaks at 2θ in angular degrees using Cu—Kα radiation:

about 3.39,

about 12.85,

about 13.42,

about 17.56,

about 18.69,

about 21.02, and

about 21.47.

In a fifth embodiment, the present invention provides crystal form D which has the following characteristic diffraction peaks at 2θ in angular degrees using Cu—Kα radiation:

about 3.39,

about 12.85,

about 13.42,

about 17.56,

about 18.69,

about 21.02,

about 21.47, and

about 23.33.

In a sixth embodiment, the present invention provides crystal form D which has the following characteristic diffraction peaks at 2θ in angular degrees using Cu—Kα radiation:

about 3.39,

about 12.85,

about 13.42,

about 17.56,

about 18.69,

about 21.02,

about 21.47,

about 23.33, and

about 24.82.

In a seventh embodiment, the present invention provides crystal form D which has the following characteristic diffraction peaks at 2θ in angular degrees using Cu—Kα radiation:

about 3.39,

about 6.54,

about 12.85,

about 13.42,

about 17.56,

about 18.69,

about 21.02,

about 21.47,

about 23.33, and

about 24.82.

In an eighth embodiment, the present invention provides crystal form D which has the following characteristic diffraction peaks at 2θ in angular degrees using Cu—Kα radiation:

about 3.39,

about 6.54,

about 8.32,

about 10.49,

about 12.16,

about 12.85,

about 13.42,

about 14.79,

about 16.44,

about 17.56,

about 18.69,

about 21.02,

about 21.47,

about 23.33,

about 24.82,

about 26.73,

about 28.43,

about 29.85,

about 33.80, and

about 37.73.

In the context of the position of the diffraction peaks in °2θ, the term about means ±0.2°.

In another embodiment, the present invention provides crystal form D which has the following characteristic diffraction peaks at 2θ in angular degrees using Cu—Kα radiation and the following relative intensities:

| Peak Position (°2θ) | Relative Intensity (%) |
|---|---|
| about 3.39 | 100 |
| about 13.42 | about 28 |
| about 18.69 | about 14 |
| about 21.02 | about 15 |
| about 21.47 | about 13 |

In another embodiment, the present invention provides crystal form D which has the following characteristic diffraction peaks at 2θ in angular degrees using Cu—Kα radiation and the following relative intensities:

| Peak Position (°2θ) | Relative Intensity (%) |
|---|---|
| about 3.39 | 100 |
| about 12.85 | about 12 |
| about 13.42 | about 28 |
| about 18.69 | about 14 |
| about 21.02 | about 15 |
| about 21.47 | about 13 |

In another embodiment, the present invention provides crystal form D which has the following characteristic diffraction peaks at 2θ in angular degrees using Cu—Kα radiation and the following relative intensities:

| Peak Position (°2θ) | Relative Intensity (%) |
|---|---|
| about 3.39 | 100 |
| about 12.85 | about 12 |
| about 13.42 | about 28 |
| about 16.44 | about 10 |
| about 18.69 | about 14 |
| about 21.02 | about 15 |
| about 21.47 | about 13 |

In another embodiment, the present invention provides crystal form D which has the following characteristic diffraction peaks at 2θ in angular degrees using Cu—Kα radiation and the following relative intensities:

| Peak Position (°2θ) | Relative Intensity (%) |
|---|---|
| about 3.39 | 100 |
| about 12.85 | about 12 |
| about 13.42 | about 28 |
| about 16.44 | about 10 |
| about 18.69 | about 14 |
| about 21.02 | about 15 |
| about 21.47 | about 13 |
| about 23.33 | about 10 |

In another embodiment, the present invention provides crystal form D which has the following characteristic diffraction peaks at 2θ in angular degrees using Cu—Kα radiation and the following relative intensities:

| Peak Position (°2θ) | Relative Intensity (%) |
|---|---|
| about 3.39 | 100 |
| about 12.85 | about 12 |
| about 13.42 | about 28 |
| about 16.44 | about 10 |
| about 18.69 | about 14 |
| about 21.02 | about 15 |
| about 21.47 | about 13 |
| about 23.33 | about 10 |
| about 24.82 | about 8 |

In another embodiment, the present invention provides crystal form D which has the following characteristic diffraction peaks at 2θ in angular degrees using Cu—Kα radiation and the following relative intensities:

| Peak Position (°2θ) | Relative Intensity (%) |
|---|---|
| about 3.39 | 100 |
| about 6.54 | about 7 |
| about 12.85 | about 12 |
| about 13.42 | about 28 |
| about 16.44 | about 10 |
| about 18.69 | about 14 |
| about 21.02 | about 15 |
| about 21.47 | about 13 |
| about 23.33 | about 10 |
| about 24.82 | about 8 |

In another embodiment, the present invention provides crystal form D which has the following characteristic diffraction peaks at 2θ in angular degrees using Cu—Kα radiation and the following relative intensities:

| Peak Position (°2θ) | Relative Intensity (%) |
|---|---|
| about 3.39 | 100 |
| about 6.54 | about 7 |
| about 8.32 | about 5 |
| about 10.45 | about 2 |
| about 12.16 | about 3 |
| about 12.85 | about 12 |
| about 13.42 | about 28 |
| about 14.79 | about 2 |
| about 16.44 | about 10 |
| about 17.56 | about 2 |
| about 18.69 | about 14 |
| about 21.02 | about 15 |
| about 21.47 | about 13 |
| about 23.33 | about 10 |
| about 24.82 | about 8 |
| about 26.73 | about 3 |
| about 28.43 | about 3 |
| about 29.85 | about 5 |
| about 33.80 | about 1 |
| about 37.73 | about 1 |

In the context of relative intensity, the term “about” means ±2%.

Crystal form D shows stable features under several conditions of relative humidity, as demonstrated in Example 2 below.

The present invention also provides processes for the preparation of said polymorph which comprise crystallizing raw CHF 4226 from a solution thereof in a solvent or a mixture of solvents under conditions which yield said crystal form. The precise conditions under which said form is obtained may be empirically determined and it is only possible to give a number of methods which have been found to be suitable in practice.

In general the polymorphic form of the invention may be prepared by crystallization under particular conditions of raw CHF 4226 obtained as reported in EP 147719 or by re-crystallization of the previously isolated crystal form A or by re-crystallization of any other crystal forms which may become known in the future.

Thus, for example, crystal form D may be prepared by crystallization from a solvent kept at the refluxing temperature (55 to 80° C.).

In general, a solvent in which the solubility of CHF 4226 is low, e.g. acetone, acetonitrile or tetrahydrofuran, is used. In order to dissolve the starting material, it may be helpful to add an amount of water or methanol up to 15% v/v in the solvent system. The solution is then cooled to a temperature of 0 to 5° C. at a cooling rate comprised between 5° and 10° C./hour, preferably 10° C./hour, in order to precipitate D crystals out from the solution.

The polymorph of the present invention is readily isolable and it exhibits favorable filtration characteristics. Optionally, it may be filtered off from the crystallization medium, after washing and drying.

The crystal form of the present invention can be desiccated under gentle temperature conditions, and after drying it shows a low amount of residual solvents, typically less than 0.5% by weight.

If desired, crystal form D prepared as above may further be re-crystallized using crystallization conditions similar to those described above.

For subsequent crystallizations, it may be preferable to add a “seed” of the crystalline material to the solution in order to induce crystallization.

The polymorph of the present invention may be formulated for administration in any convenient way, and the present invention also provides pharmaceutical compositions comprising CHF 4226 crystal form D.

The skilled person can establish without undue experimentation the effective dosage and/or the concentration of the active ingredient in the composition for a specific therapeutic purpose. In the compositions provided herein, an effective concentration of the polymorph of the invention is mixed with one or more suitable pharmaceutical carriers or vehicles or excipients, for example those described in Remington's Pharmaceutical Sciences Handbook, Mack. Pub., N.Y., USA.

The concentration of the polymorph in the formulation is effective for delivery, upon administration, an amount sufficient to exert a therapeutically useful effect.

The compositions may also contain, if required, one or more other therapeutic agents, preferably those currently used in the treatment of respiratory disorders, e.g. corticosteroids such as budesonide and its epimers, beclometasone dipropionate, triamcinolone acetonide, fluticasone propionate, flunisolide, mometasone furoate, rofleponide and ciclesonide, anticholinergic or antimuscarinic agents such as ipratropium bromide, oxytropium bromide, tiotropium bromide, glycopyrrolate bromide, and the group of phosphodiesterase-4 (PDE-4) inhibitors such as roflumilast.

The polymorph of the invention may be formulated for oral, buccal, topical, parenteral, vaginal, rectal, or inhalation administration. Inhalation administration is particularly preferred.

With the inhalation route of administration, the dose regimen is twice or once daily, where the suitable dose is advantageously in the range of 0.5 to 8 μg, preferably of 1 to 4 μg, more preferably of 2 to 4 μg.

Inhalable preparations include inhalable powders, propellant-containing metering aerosols or propellant-free inhalable solution or suspension formulations.

Advantageously, the inhalable powder formulations for inhalation comprise the polymorph of the invention in the form of interactive ordered mixtures.

More advantageously said formulations comprise a fraction of coarse particles of a physiologically acceptable excipient such as alpha-lactose monohydrate, said particles having a mass median diameter (MMD) higher than 90 micron, preferably the mass diameter (MD) comprised between 50 micron and 500 micron, more preferably between 150 and 400 micron, even more preferably between 210 and 355 micron.

Preferably said powder formulations further comprise a fraction of microparticles, obtained by co-milling, having a MMD lower than 35 micron, and constituted of particles of a physiologically acceptable excipient and an additive material selected from the class of the anti-adherents such as the amino acids leucine and isoleucine or of the lubricants such as magnesium stearate; sodium stearyl fumarate stearyl alcohol, stearic acid and sucrose monopalmitate.

More preferably said powder formulations comprise a fraction of microparticles having a MMD lower than 15 micron, preferably lower than 10 micron, constituted of particles of alpha-lactose monohydrate and particles of magnesium stearate.

Even more preferably, the inhalable powder formulations comprising the polymorph of the invention are prepared according to the teaching of co-pending application PCT/IB2007/0038924.

In general, CHF 4226 crystal form D may be used in preparation of a medicament for any disease or condition in which it is found therapeutically effective.

Having regard to its beta₂-adrenoceptor stimulating activity, CHF 4226 crystal form D is useful in the relaxation of bronchial smooth muscle and the relief of bronchoconstriction. Relief of bronchoconstriction can be measured in models such as the in vivo guinea pigs model (see Kikkawa et al., Biol. Pharm. Bull., 1994, 17(8), 1047-1052) and analogous models.

Administration of the polymorph of the invention may be indicated for the prevention and/or treatment of mild, moderate, or severe, acute or chronic symptoms, or for prophylactic treatment of obstructive respiratory diseases such as asthma and chronic obstructive pulmonary disease (COPD). Other respiratory disorders characterized by obstruction of the peripheral airways as a result of inflammation and presence of mucus such as chronic obstructive bronchiolitis and chronic bronchitis may also benefit from their use.

Other features of the invention will become apparent in the course of the following descriptions of exemplary embodiments which are given for illustration of the invention and are not intended to be limiting thereof.

### EXAMPLES

Example 1 deals with the preparation of crystal form D, Example 2 deals with stability experiments, Example 3 refers to an exemplary formulation, and Comparative Example 4 refers to the comparative characteristics of crystal form A.

### Example 1

### Preparation of CHF 4226 Crystal Form D by Crystallization

Crystallization was performed in a 500 ml jacketed glass reactor connected to thermocryostat, so that temperature could be accurately controlled; in addition, crystallization was followed using a Lasentec FBRM D600 probe, so that nucleation and crystals growth could be monitored. 30 g of raw CHF4226 obtained as reported in EP 147719 was suspended in 250 ml of acetonitrile at refluxing temperature (55° C.), and then 36 ml of water (87:13 v/v) was added portion-wise until dissolution was complete. The solution was cooled to a temperature of 0 to 5° C. at a cooling rate of 10° C./minute. After 20 hours, a solid sample was collected, filtered, dried at T=40° C. under vacuum, and analyzed for solid state, e.g. by XRPD and DSC.

### Methods of Analysis:

### 1. X-ray Powder Diffraction (XRPD).

The XRPD analyses were carried out on a PANanalytical X'pert Pro X-ray powder diffractometer using Cu Kα radiation. The instrument was equipped with a X'Celerator detector. A theta-two theta continuous scan from 2.5 degrees 2 theta to 40 degrees 2 theta was used. Each sample was prepared for analysis by placing it in a quartz sample holder. The XPRD patterns are reported in terms of degrees 2 theta (°2θ, accuracy ±0.1°), diffraction peak intensity (height, [cts]), full width at half maximum (FWHM, [°2θ]), interatomic spacing in angstroms (d-spacing, [Å]), and relative intensities (%). The relative intensity is recorded as the ratio of the peak intensity to that of the most intense peak.

### 2. Differential Scanning Calorimetry (DSC).

The differential scanning calorimetry data were obtained on a Diamon Perkin Elmer Instrument. The calibration standard used was indium. Approximately 2 to 5 mg of a sample was placed into a DSC pan, and the weight was accurately measured and recorded. The pan was hermetically sealed. The sample was heated under nitrogen at a rate of 10° C./minute, from 25° C. to a final temperature of 200° C.

The results of the DSC and XRPD analysis are shown in FIGS. 1 and 2.

Yield: 12.4 g (41.3%).

Purity (HPLC): >99%.

### Example 2

### Stability Studies

In order to check its thermodynamic stability, CHF 4226 crystal form D was stored at room temperature under different relative humidity (RH) conditions:
40% RH. and 25° C. for 1 week
60% RH. and 25° C. for 1 week
80% RH and 25° C. for 1 week
60% RH and 25° C. for 1 week
40% RH and 25° C. for 1 week
30% RH and 25° C. for 1 week
0% RH. and 25° C. for 1 week
40% RH and 25° C. for 1 week
   The thermodynamic stability of all the samples was checked by recording their XPRD pattern. Crystal form D showed stable features under all stability screening conditions.

### Example 3

### Exemplary Inhalable Dry Powder Formulation Comprising CHF 4226 Crystal Form D

The composition is reported in Table 2.

TABLE 2

| | Amounts | | |
|---|---|---|---|
| | Per shot of | | |
| | the inhaler | | Single dose |
| Components | mg | % | μg |
| CHF 4226 crystal form D | 0.004 | 0.04 | 4 |
| alpha-lactose monohydrate 212-355 μm | 8.996 | 89.96 | |
| microparticles of alpha-lactose | 1.00 | 10.0 | |
| monohydrate and magnesium stearate | | | |
| obtained by co-milling | | | |
| Total weight | 10 | | |

### Comparative Example 4

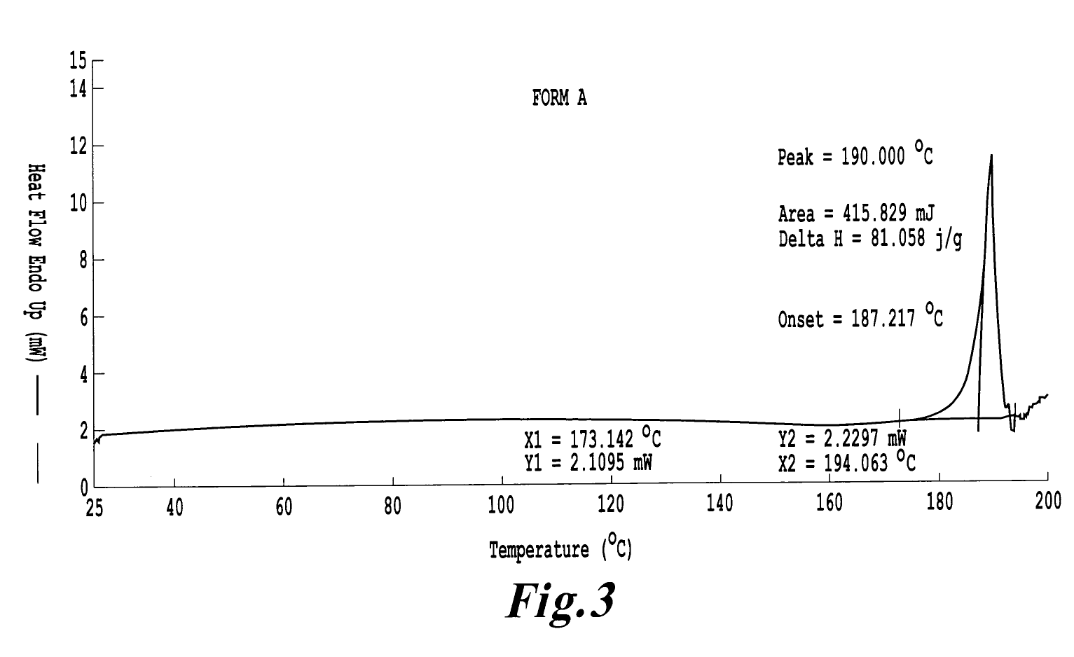

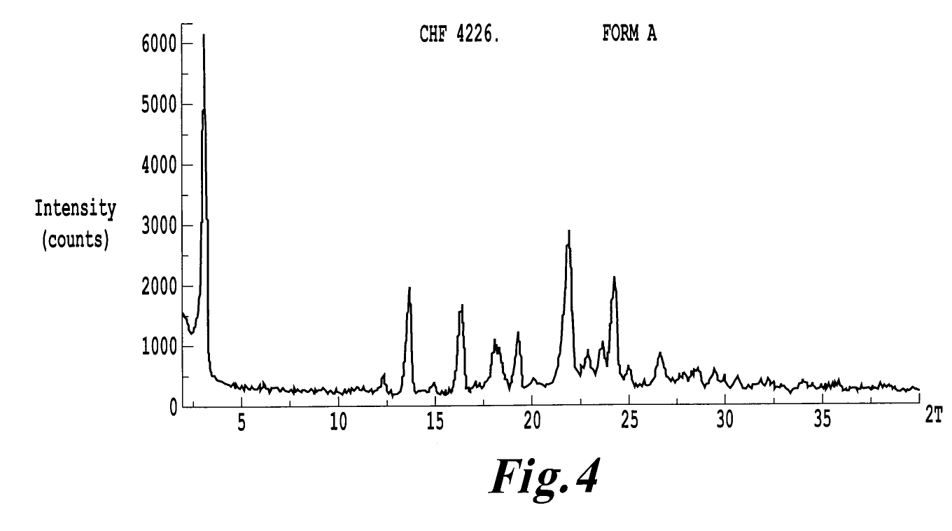

CHF 4226 crystal form A was prepared according to the Example of pending application WO 2005/089760. Its thermal trace, shown in FIG. 3, exhibits a melting peak at 190.0° C. Crystal form A has the characteristic diffraction lines in the XPRD pattern thereof shown in FIG. 4. Said characteristic diffraction lines are reported in Table 3.

TABLE 3

| Pos. [°2θ] | Height [cts] | FWHM [°2θ] | d-spacing [Å] | Rel. Int. [%] |
|---|---|---|---|---|
| 3.2171 | 4882.25 | 0.1673 | 27.46407 | 100.00 |
| 6.1648 | 56.30 | 0.2007 | 14.33719 | 1.15 |
| 11.0017 | 48.41 | 0.5353 | 8.04228 | 0.99 |
| 12.2932 | 221.92 | 0.2007 | 7.20012 | 4.55 |
| 13.6848 | 1628.41 | 0.2007 | 6.47093 | 33.35 |
| 14.9235 | 119.56 | 0.2007 | 5.93650 | 2.45 |
| 16.4033 | 1404.06 | 0.2007 | 5.40410 | 28.76 |
| 17.0392 | 72.21 | 0.2676 | 5.20383 | 1.48 |
| 18.0629 | 735.52 | 0.1673 | 4.91115 | 15.07 |
| 18.3442 | 579.41 | 0.1673 | 4.83647 | 11.87 |
| 19.4085 | 652.26 | 0.0836 | 4.57360 | 13.36 |
| 20.0887 | 115.52 | 0.2007 | 4.42025 | 2.37 |
| 22.0423 | 1972.56 | 0.2175 | 4.03270 | 40.40 |
| 22.9299 | 403.55 | 0.1171 | 3.87856 | 8.27 |
| 23.6648 | 552.77 | 0.2007 | 3.75976 | 11.32 |
| 24.3181 | 1607.95 | 0.2175 | 3.66022 | 32.93 |
| 25.0090 | 237.34 | 0.1673 | 3.56064 | 4.86 |
| 26.6661 | 452.07 | 0.3346 | 3.34302 | 9.26 |
| 28.6123 | 224.79 | 0.4015 | 3.11990 | 4.60 |
| 29.5322 | 204.45 | 0.2342 | 3.02478 | 4.19 |
| 30.6166 | 147.68 | 0.2676 | 2.92007 | 3.02 |
| 32.2879 | 129.66 | 0.2007 | 2.77264 | 2.66 |
| 33.9670 | 131.82 | 0.2676 | 2.63932 | 2.70 |
| 35.8516 | 103.81 | 0.2676 | 2.50479 | 2.13 |
| 38.2891 | 50.71 | 0.6691 | 2.35076 | 1.04 |

The results indicate that crystal form D is clearly distinguishable from crystal form A of CHF 4226.

### Comparative Example 5

CHF 4226 is also prepared according to the Example 4 of EP 147719. The compound is obtained in a prevalently amorphous. However the diffraction lines in the XPRD pattern, reported in Table 4, establish that the obtained form is different and clearly distinguishable from crystal form D.

TABLE 4

| Pos. [°2θ] | Height [cts] | FWHM [°2θ] | d-spacing [Å] | Rel. Int. [%] |
|---|---|---|---|---|
| 3.2301 | 6070.87 | 0.1171 | 27.35334 | 100.00 |
| 6.2308 | 45.98 | 0.4015 | 14.18532 | 0.76 |
| 9.2012 | 39.49 | 0.4015 | 9.61152 | 0.65 |
| 12.3369 | 153.61 | 0.2007 | 7.17472 | 2.53 |
| 13.6352 | 1192.46 | 0.1171 | 6.49434 | 19.64 |
| 16.1071 | 311.73 | 0.2007 | 5.50282 | 5.13 |
| 16.8444 | 240.05 | 0.1338 | 5.26359 | 3.95 |
| 18.0251 | 426.10 | 0.2676 | 4.92137 | 7.02 |
| 18.7917 | 650.30 | 0.2007 | 4.72230 | 10.71 |
| 20.4675 | 57.55 | 0.4015 | 4.33929 | 0.95 |
| 21.4611 | 663.40 | 0.2007 | 4.14057 | 10.93 |
| 21.8671 | 918.91 | 0.1171 | 4.06462 | 15.14 |
| 22.8287 | 170.10 | 0.2676 | 3.89553 | 2.80 |
| 23.4886 | 410.61 | 0.2342 | 3.78756 | 6.76 |
| 24.6559 | 364.08 | 0.5353 | 3.61082 | 6.00 |
| 26.4968 | 90.38 | 0.5353 | 3.36400 | 1.49 |
| 28.3431 | 278.73 | 0.1338 | 3.14892 | 4.59 |
| 30.1299 | 111.82 | 0.4015 | 2.96613 | 1.84 |
| 32.2904 | 57.48 | 0.5353 | 2.77243 | 0.95 |
| 33.9931 | 76.27 | 0.2676 | 2.63736 | 1.26 |

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. Crystalline 8-hydroxy-5-[(1R)-1-hydroxy-2-[[(1R)-2-(4-methoxyphenyl)-1-methylethyl]amino]ethyl]-2(1H)-quinolinone monohydrochloride having diffraction peaks at 2θ in angular degrees using Cu—Kα radiation at:
about 3.39,
about 13.42,
about 18.69,
about 21.02, and
about 21.47.

2. Crystalline 8-hydroxy-5-[(1R)-1-hydroxy-2-[[(1R)-2-(4-methoxyphenyl)-1-methylethyl]amino]ethyl]-2(1H)-quinolinone monohydrochloride according to claim 1, which has diffraction peaks at 2θ in angular degrees using Cu—Kα radiation at:
about 3.39,
about 12.85,
about 13.42,
about 18.69,
about 21.02, and
about 21.47.

3. Crystalline 8-hydroxy-5-[(1R)-1-hydroxy-2-[[(1R)-2-(4-methoxyphenyl)-1-methylethyl]amino]ethyl]-2(1H)-quinolinone monohydrochloride according to claim 1, which has diffraction peaks at 20 in angular degrees using Cu—Kα radiation at:
about 3.39,
about 12.85,
about 13.42,
about 17.56,
about 18.69,
about 21.02, and
about 21.47.

4. A process for preparing crystalline 8-hydroxy-5-[(1R)-1-hydroxy-2-[[(1R)-2-(4-methoxyphenyl)-1-methylethyl]amino]ethyl]-2(1H)-quinolinone monohydrochloride according to claim 1, which comprises:
crystallizing 8-hydroxy-5-[(1R)-1-hydroxy-2-[[(1R)-2-(4-methoxyphenyl) 1-methylethyl]amino]ethyl]-2(1H)-quinolinone monohydrochloride from a solution thereof in a solvent selected from the group consisting of acetone, acetonitrile, tetrahydrofuran, and mixtures thereof kept at a refluxing temperature.

5. A process according to claim 4, wherein said solution is cooled to a temperature of 0 to 5° C. at a cooling rate comprised between 5 and 10° C./minute.

6. A pharmaceutical composition, comprising crystalline 8-hydroxy-5-[(1R)-1-hydroxy-2-[[(1R)-2-(4-methoxyphenyl)-1-methylethyl]amino]ethyl]-2(1H)-quinolinone monohydrochloride according to claim 1 and at least one pharmaceutically acceptable carrier.

7. A pharmaceutical composition according to claim 6, further comprising at least one therapeutic agent selected from the group consisting of a corticosteroid, an anticholinergic agent, an antimuscarinic agent, and a phosphodiesterase-4 (PDE-4) inhibitor.

8. A pharmaceutical composition according to claim 6, which is in the form of an inhalable aerosol comprising a propellant.

9. A pharmaceutical composition according to claim 6, which is in the form of an inhalable powder.

10. A method for the prevention and/or treatment of an inflammatory or obstructive respiratory disease, comprising administering an effective amount of crystalline 8-hydroxy-5-[(1R)-1-hydroxy-2-[[(1R)-2-(4-methoxyphenyl)-1-methylethyl]amino]ethyl]-2(1H)-quinolinone monohydrochloride according to claim 1, to a subject in need thereof.

11. The method of claim 10, wherein said respiratory disease is asthma or chronic obstructive pulmonary disease.

12. A pharmaceutical composition, comprising crystalline 8-hydroxy-5-[(1R)-1-hydroxy-2-[[(1R)-2-(4-methoxyphenyl)-1-methylethyl]amino]ethyl]-2(1H)-quinolinone monohydrochloride according to claim 2 and at least one pharmaceutically acceptable carrier.

13. A pharmaceutical composition according to claim 12, further comprising at least one therapeutic agent selected from the group consisting of a corticosteroid, an anticholinergic agent, an antimuscarinic agent, and a phosphodiesterase-4 (PDE-4) inhibitor.

14. A pharmaceutical composition according to claim 12, which is in the form of an inhalable aerosol comprising a propellant.

15. A pharmaceutical composition according to claim 12, which is in the form of an inhalable powder.

16. A method for the prevention and/or treatment of an inflammatory or obstructive respiratory disease, comprising administering an effective amount of crystalline 8-hydroxy-5-[(1R)-1-hydroxy-2-[[(1R)-2-(4-methoxyphenyl)-1-methylethyl]amino]ethyl]-2(1H)-quinolinone monohydrochloride according to claim 2 to a subject in need thereof.

17. The method of claim 16, wherein said respiratory disease is asthma or chronic obstructive pulmonary disease.
